(19) United States
(12) Patent Application Publication
(10) Pub. No.: US 2004/0067227 A1
(43) Pub. Date: Apr. 8, 2004

(54) Inhibition or reversal of skin aging by actin-sequestering peptides

(76) Inventor: Allan L. Goldstein, Washington, DC (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/415,407

(22) PCT Filed: Nov. 2, 2001
(86) PCT No.: PCT/US01/42900

(51) Int. Cl. A61K 39/395; A61K 38/17

(52) U.S. Cl. 424/130.1; 514/12

(57) ABSTRACT

Skin degradation associated with skin aging is inhibited or reversed by administration of an actin-sequestering peptide such as Thymosin β4, an isoform of Thymosin β4 or oxidized Thymosin β4.

### CROSS-REFERENCE TO RELATED APPLICATION

The present application claims the benefit of U.S. Provisional Application Serial No. 60/244,901, filed Nov. 2, 2000.

### BACKGROUND OF THE INVENTION

1. Field of the Invention

The present invention relates to the field of inhibiting or reversing skin aging.

2. Description of the Background Art

The phenomenon called skin “aging” may occur not only with advancing age, but due to other degenerative changes and environmental factors. Skin aging results from deleterious changes in the physiological, biochemical and immunological properties of the skin. Such changes include thinning of the skin, loss of elasticity, alteration in polymerized actin ratios and turnover of polymerized actin, decrease in collagen and other matrix proteins, changes in vasculature which decrease capacity to repair DNA damage, increased propensity for skin cancers such as squamous cell carcinoma, and increased risk of infection.

Numerous pharmaceutical, nutriceutical or cosmeceutical formulations have been proposed to reduce or reverse skin aging or the appearance of skin aging. In addition, chemical peels, phototherapies and various forms of plastic surgery have been proposed.

There remains a need in the art for improved methods and compositions for inhibiting or reversing skin aging.

### SUMMARY OF THE INVENTION

In accordance with the present invention, a method of treatment for promoting reversal of or inhibiting skin degeneration associated with skin aging involves administration to a subject or patient in need of such treatment an effective amount of a composition comprising a skin degeneration-inhibiting polypeptide comprising amino acid sequence LKKTET or a conservative variant thereof having skin degeneration-inhibiting activity.

### DETAILED DESCRIPTION OF THE INVENTION

The present invention is based on a discovery that actin-sequestering peptides such as thymosin β4 (Tβ4) and other actin-sequestering peptides containing amino acid sequence LKKTET or conservative variants thereof, promote reversal of or inhibit skin degeneration associated with skin aging.

Thymosin β4 was-initially identified as a protein that is up regulated during endothelial cell migration and differentiation in vitro. Thymosin β4 was originally isolated from the thymus and is a 43 amino acid, 4.9 kDa ubiquitous polypeptide identified in a variety of tissues. Several roles have been ascribed to this protein including a role in a endothelial cell differentiation and migration, T cell differentiation, actin sequestration and vascularization.

In accordance with one embodiment, the invention is a method of treatment for promoting reversal of or inhibiting skin degradation associated with skin aging comprising administering to a subject in need of such treatment an effective amount of a composition comprising an agent that stimulates production of a skin degeneration-inhibiting polypeptide comprising amino acid sequence LKKTET, or a conservative variant thereof having skin degeneration-inhibiting activity, preferably Thymosin β4, an isoform of Thymosin β4, oxidized Thymosin β4 or an antagonist of Thymosin β4.

The present invention promotes skin condition improvements selected from the group consisting of an increase in skin elasticity, size reduction of an area of age-related skin darkening (age spots), lightening of an area of age-related skin darkening, and combinations thereof.

Compositions which may be used in accordance with the present invention include Thymosin β4 (Tβ4), Tβ4 isoforms, oxidized Tβ4, polypeptides comprising the amino acid sequence LKKTET or conservative variants thereof having skin degeneration-inhibiting activity. International Application Serial No. PCT/US99/17282, incorporated herein by reference, discloses isoforms of Tβ4 which may be useful in accordance with the present invention as well as amino acid sequence LKKTET and conservative variants thereof having skin degeneration-inhibiting activity, which may be utilized with the present invention. International Application Serial No. PCT/GB99/00833 (WO 99/49883), incorporated herein by reference, discloses oxidized Thymosin β4 which may be utilized in accordance with the present invention. Although the present invention is described primarily hereinafter with respect to Tβ4 and Tβ4 isoforms, it is to be understood that the following description is intended to be equally applicable to amino acid sequence LKKTET, conservative variants thereof having skin degeneration-inhibiting activity, as well as oxidized Thymosin β4.

In one embodiment, the invention provides a method for inhibiting or reversing aging of skin in a subject by contacting the skin with a skin degeneration-inhibiting effective amount of a composition which contains Tβ4 or a Tβ4 isoform. The contacting may be topically or systemically. Examples of topical administration include, for example, contacting the skin with a lotion, salve, gel, cream, paste, spray, suspension, dispersion, hydrogel, ointment, or oil comprising Tβ4. Systemic administration includes, for example, intravenous, intraperitoneal, intramuscular injections of a composition containing Tβ4 or a Tβ4 isoform. A subject may be any mammal, preferably human.

A composition in accordance with the present invention can be administered daily, every other day, etc., with a single application or multiple applications per day of administration, such as applications 2, 3, 4 or more times per day of administration.

Tβ4 isoforms have been identified and have about 70%, or about 75%, or about 80% or more homology to the known amino acid sequence of Tβ4. Such isoforms include, for example, to tβ4^{ala}, Tβ9, Tβ10, Tβ11, Tβ12, Tβ13, Tβ14 and Tβ15. Similar to Tβ4, the Tβ10 and Tβ15 isoforms have been shown to sequester actin. Tβ4, Tβ10 and Tβ15, as well as these other isoforms share an amino acid sequence, LKKTET, that appears to be involved in mediating actin sequestration or binding. Although not wishing to be bound to any particular theory, the activity of Tβ4 isoforms may be due, in part, to the ability to polymerize actin. For example, Tβ4 can modulate actin polymerization in skin (e.g. β-thymosins appear to depolymerize F-actin by sequestering free G-actin). Tβ4's ability to modulate actin polymerization may therefore be due to all, or in part, its ability to bind to or sequester actin via the LKKTET sequence. Thus, as with Tβ4, other proteins which bind or sequester actin, or modulate actin polymerization, including Tβ4 isoforms having the amino acid sequence LKKTET, are likely to reduce skin aging, alone or in a combination with Tβ4, as set forth herein.

Thus, it is specifically contemplated that known Tβ4 isoforms, such as Tβ4^{ala}, Tβ9, Tβ10, Tβ11, Tβ12, Tβ13, Tβ14 and Tβ15, as well as Tβ4 isoforms not yet identified, will be useful in the methods of the invention. As such Tβ4 isoforms are useful in the methods of the invention, including the methods practiced in a subject. The invention therefore further provides pharmaceutical compositions comprising Tβ4, as well as Tβ4 isoforms Tβ4^{ala}, Tβ9, Tβ10, Tβ11, Tβ12, Tβ13, Tβ14 and Tβ15, and a pharmaceutically acceptable carrier.

In addition, other proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET, for example, can similarly be employed in the methods of the invention. Such proteins include gelsolin, vitamin D binding protein (DBP), profilin, cofilin, depactin, DnaseI, vilin, fragmin, severin, capping protein, β-actinin and acumentin, for example. As such methods include those practiced in a subject, the invention further provides pharmaceutical compositions comprising gelsolin, vitamin D binding protein (DBP), profilin, cofilin, depactin, DnaseI, vilin, fragmin, severin, capping protein, β-actinin and acumentin as set forth herein. Thus, the invention includes the use of a skin aging reducing polypeptide comprising the amino acid sequence LKKTET and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

Tβ4 has been localized to a number of tissue and cell types and thus, agents which stimulate the production of Tβ4 can be added to or comprise a composition to effect Tβ4 production from a tissue and/or a cell. Such agents include members of the family of growth factors, such as insulin-like growth factor (IGF-1), platelet derived growth factor (PDGF), epidermal growth factor (EGF), transforming growth factor beta (TGF-β), basic fibroblast growth factor (bFGF), thymosin α1 (Tα1) and vascular endothelial growth factor (VEGF). More preferably, the agent is transforming growth factor beta (TGF-β) or other members of the TGF-β superfamily. Tβ4 compositions of the invention may reduce skin aging by effectuating growth of the connective tissue through extracellular matrix deposition, cellular migration and vascularization of the skin.

Additionally, agents that assist or stimulate skin aging reduction maybe added to a composition along with Tβ4 or a Tβ4 isoform. Such agents include angiogenic agents, growth factors, agents that direct differentiation of cells, agents that promote migration of cells and agents that stimulate the provision of extracellular matrix material in the skin. For example, and not by way of limitation, Tβ4 or a Tβ4 isoform alone or in combination can be added in combination with any one or more of the following agents: VEGF, KGF, FGF, PDGF, TGFβ, IGF-1, IGF-2, IL-1, prothymosin α and thymosin α1 in an effective amount.

The invention also includes a pharmaceutical composition comprising a therapeutically effective amount of Tβ4 or a Tβ4 isoform in a pharmaceutically acceptable carrier. Such carriers include those listed above with reference to parenteral administration.

The actual dosage or reagent, formulation or composition that inhibits or promotes reversal of skin aging may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use.

Suitable topical formulations include Tβ4 or a Tβ4 isoform at a concentration within the range of about 0.001-10% by weight, more preferably within the range of about 0.01-0.1% by weight, most preferably about 0.05% by weight.

The therapeutic approaches described herein involve various routes of administration or delivery of reagents or compositions comprising the Tβ4 or other compounds of the invention, including any conventional administration techniques (for example, but not limited to, topical administration, local injection, inhalation, or systemic administration), to a subject. The methods and compositions using or containing Tβ4 or other compounds of the invention may be formulated into pharmaceutical compositions by admixture with pharmaceutically acceptable non-toxic excipients or carriers.

The invention includes use of antibodies which interact with Tβ4 peptide or functional fragments thereof. Antibodies which consists essentially of pooled monoclonal antibodies with different epitopic specificities, as well as distinct monoclonal antibody preparations are provided. Monoclonal antibodies are made from antigen containing fragments of the protein by methods well known to those skilled in the art as disclosed in PCT/US99/17282, supra. The term antibody as used in this invention is meant to include monoclonal and polyclonal antibodies.

In yet another embodiment, the invention provides a method of treating a subject by administering an effective amount of an agent which modulates Tβ4 gene expression. The term “modulate” refers to inhibition or suppression of Tβ4 expression when TP is over expressed, and induction of expression when Tβ4 is under expressed. The term “effective amount” means that amount of Tβ4 agent which is effective in modulating Tβ4 gene expression resulting in reducing the symptoms of the Tβ4 associated skin aging. An agent which modulates Tβ4 or Tβ4 isoform gene expression may be a polynucleotide for example. The polynucleotide may be an antisense, atriplex agent, or a ribozyme. For example, an antisense directed to the structural gene region or to the promoter region of Tβ4 may be utilized.

In another embodiment, the invention provides a method for utilizing compounds that modulate Tβ4 activity. Compounds that affect Tβ4 activity (e.g., antagonists and agonists) include peptides, peptidomimetics, polypeptides, chemical compounds, minerals such as zincs, and biological agents.

While not be bound to any particular theory, it is believed that the present invention may promote reversal of or inhibit skin degeneration associated with skin aging by inducing terminal deoxynucleotidyl transferase (a non-template directed DNA polymerase), to decrease the levels of one or more inflammatory cytokines, and to act as a chemotactic factor for endothelial cells, and thereby inhibit or promote reversal of degenerative changes in skin brought about by aging or other degenerative or environmental factors.

The invention is further illustrated by the following non-limiting example.

### EXAMPLE 1

A 0.05% by weight Thymosin β4 formulation was prepared, i.e., 50 mg Thymosin 4 per 100 gm gel, by first dissolving Thymosin β4 in water and thoroughly mixing the preparation in a standard pharmaceutical grade gel formulation. A volunteer with a dark 1 cm age spot on the dorsal region of the hand below the middle knuckle was treated. The 0.05% by weight Thymosin β4 gel was applied to a 5×5 cm region encompassing the age spot, twice daily for 28 days. Within seven days the age spot began to fade and within 14 days, the age spot began to noticeably decrease in size. At the end of the 28 day period, the age spot had faded significantly and the diameter of the spot decreased by over 50%. Additionally, the skin in the treated area became smoother and appeared to have increased elasticity. The volunteer was subsequently observed for four weeks, and the changes-observed during treatment persisted.

1. A method of treatment for promoting reversal of or inhibiting skin degeneration associated with skin aging, comprising administering to a subject in need of such treatment an effective amount of a composition comprising a skin degeneration-inhibiting polypeptide comprising amino acid sequence LKKTET, or a conservative variant thereof having skin degeneration-inhibiting activity.

2. The method of claim 1 wherein said polypeptide promotes a skin condition improvement selected from the group consisting of an increase in skin elasticity, size reduction of an area of age-related skin darkening, lightening of an area of age-related skin darkening, and combinations thereof.

3. The method of claim 1 wherein said polypeptide comprises Thymosin β4 (Tβ4), an isoform of Tβ4 or oxidized Tβ4.

4. The method of claim 1 wherein said composition is administered systemically.

5. The method of claim 1 wherein said composition is administered topically.

6. The method of claim 5 wherein said composition is in the form of a gel, creme, paste, lotion, spray, suspension, dispersion, salve, hydrogel or ointment formulation.

7. The method of claim 1 wherein said polypeptide is recombinant or synthetic.

8. The method of claim 1 wherein said polypeptide is an antibody.

9. The method of claim 8 wherein said antibody is polyclonal or monoclonal.

10. A method of treatment for promoting reversal of or inhibiting skin degeneration associated with skin aging comprising administering to a subject in need of such treatment an effective amount of a composition comprising an agent that stimulates production of a skin degeneration-inhibiting polypeptide comprising amino acid sequence LKKTET, or a conservative variant thereof having skin degeneration-inhibiting activity.

11. The method of claim 10 wherein said polypeptide is Thymosin β4.

12. The method of claim 10 wherein said agent is an antagonist of Thymosin β4.

13. A composition for use in promoting reversal of or inhibiting skin degeneration associated with skin aging comprising an effective amount of a composition including a skin degeneration-inhibiting polypeptide comprising amino acid sequence LKKTET or a conservative variant thereof having skin degeneration-inhibiting activity.

14. The composition of claim 13 wherein said polypeptide comprises Tβ4, an isoform of Tβ4 or oxidized Tβ4.

15. The composition of claim 13, comprising a gel, creme, paste, lotion, spray, suspension, dispersion salve, hydrogel or ointment formulation.
